(19) United States
(12) Patent Application Publication
(10) Pub. No.: US 2007/0260146 A1
(43) Pub. Date: Nov. 8, 2007

(54) In vivo spectrometric inspection system

(76) Inventors: Mang Ou-Yang, Hsinchu City (TW); Wei-Wu Kuo, Hsinchu City (TW); Hsien-Ming Wu, Hsinchu City (TW); Shih-Chieh Lu, Hsinchu City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/417,174

(22) Filed: May 4, 2006

(51) Int. Cl. A61B 6/00; A61B 1/06

(52) U.S. Cl. 600/476; 600/473; 600/160

(57) ABSTRACT

The present invention discloses an in vivo spectrometric inspection system. The system of the present invention is encapsulated in a swallowable capsule. After a testee swallows the capsule, an optical system inside the capsule generates a light source to illuminate an object. The light source hits the object to excite a light, or the light source is partially absorbed and partially reflected by the object. The excited light or the reflected light is received by the optical system and sent to a spectrometric system also inside the capsule. The spectrometric system disperses the wavelength components of the excited light or the reflected light into spectra, and then, the spectrometric system further analyzes the spectra to obtain a spectrum data. The spectrum data is sent out to the exterior of the testee body by a data-transmission device and received by an external receiver system. Otherwise, the spectrum data may be stored in a storage device inside the capsule, and after the capsule is excreted out, the spectrum data can be obtained too. Therefore, the in vivo spectrometric inspection system disclosed in the present invention can provide the information of the full-waveband spectral responses of the inspected object and promote the resolution of diagnostic inspections.

### BACKGROUND OF THE INVENTION

1. Field of the Invention

The present invention relates to an in vivo inspection system, particularly to an in vivo spectrometric inspection system, which utilizes the spectra induced by a light source to perform an in vivo diagnostic inspection.

2. Description of the Related Art

Before, the in vivo tissue inspection was usually performed with an endoscope. An endoscope, which comprises: an illuminating light source, a lens, and an image sensor, can reach the deep of the body and send out the images of in vivo tissues so that medical personnel can identify the in vivo tissues thereby.

The conventional endoscopes include: the capsular type and the optical-fiber type. Both types of the endoscopes use a white light source, i.e. the light source comprising the continuous spectrum of visible light or the light source formed via mixing at least two monochromatic light sources. However, such a white-light inspection is hard to find an abnormal tissue or an early-stage cancer, which are hard to identify with naked eyes.

A U.S. Pat. No. 5,604,531 discloses an “In Vivo Video Camera System”, which utilizes a capsule and an endoscope installed inside the capsule to obtain the image of the interior of the body for medical diagnosis. Refer to FIG. 1. The in vivo video camera system comprises a capsule 2, and the capsule 2 further comprises: a housing 4, an optical system 6, a transmitter 8, and a power source 10; the optical system 6 further comprises: a axicon element 12 having an inner hole, a light-source device 14, such as LED, a relay lens 16, and a CCD camera system 18. The capsule 2 is swallowed by a testee, and next, the light-source device 14 of the optical system 6 generates a light source to illuminate an in vivo tissue, and next, the camera system 18 captures the images of the in vivo tissue illuminated by the light source via the relay lens 16, and then, the images are sent out to an external receiving system by the transmitter 8. The abovementioned system does not adopt a waveband-selection device but only uses a white-light LED as the light source. Therefore, such a system can only obtain common white-light images of in vivo tissues.

In comparison with the video signal, the spectroscopic signal can more effectively identify a latent pathological change, especially a pre-neoplastic lesion. A nidus responds differently to different light sources, especially the autofluorescence induced by ultraviolet light; thus, the spectroscopic signal induced by a multi-waveband light source can be used to promote the discrimination rate of the early-stage inspection of abnormal cells. The abovementioned viewpoint has been proposed in numerous articles, such as “In Vivo Autofluorescence Spectroscopy of Human Bronchial Tissue to Optimize the Detection and Imaging of Early Cancers” by M. Zellweger et al., Journal of Biomedical Optics, Vol. 6, 2001, p. 45-51; and “Fluorescence Spectroscopy: a Diagnostic Tool for Cervical Intraepithelial Neoplasia (CIN)” by N. Ramanujam et al., Gynecologic Oncology, Vol. 52, 1994, p. 31-38.

Accordingly, the present invention proposes an in vivo spectrometric inspection system to overcome the above-mentioned problems.

### SUMMARY OF THE INVENTION

The primary objective of the present invention is to provide an in vivo inspection system, wherein the system is encapsulated in a swallowable capsule; after the capsule is swallowed, the capsule generates a light source to illuminate an in vivo tissue; the light source hits the tissue to excite a light, or the light source is partially absorbed and partially reflected by the tissue; the excited light or the reflected light is received to provide medical personnel to perform the succeeding inspection and analysis.

Another objective of the present invention is to provide an in vivo spectrometric inspection system, which can obtain a series of spectrum data in vivo, and medical personnel can analyze the spectrum data to obtain the tissue responses to various wavebands so that a high-accuracy diagnosis can be accomplished.

Further objective of the present invention is to provide a placed-in type capsular spectrometric endoscope, wherein generates a light source to illuminate an in vivo tissue and then receives and analyzes the spectrum of the light excited by the light source or the light reflected by the in vivo tissue and then sends out the analysis result to the exterior of the testee body.

According to one aspect of the present invention, the in vivo spectrometric inspection system of the present invention utilizes a placed-in type capsular spectrometric endoscope to generate a light source to illuminate an inspected object; the light source hits the object to excite a light, or the light source is partially absorbed and partially reflected by the object; the excited light or the reflected light is received and then analyzed spectrometrically to obtain a spectrum data; the spectrum data may be sent out to the exterior of the testee body via the following two methods: firstly, the spectrum data may be sent out by a transmitter inside the placed-in type capsular spectrometric endoscope and then received by an external receiving system; secondly, the spectrum data may be stored in a storage device inside the placed-in type capsular spectrometric endoscope and then read externally after the placed-in type capsular spectrometric endoscope is excreted from the testee body. The placed-in type capsular spectrometric endoscope comprises a swallowable capsule; the swallowable capsule further comprises: an optical system, installed inside the swallowable capsule, generating a light source to illuminate an inspected object, receiving the light excited by the light source or the light reflected by the object, and transmitting the excited light or the reflected light; a spectrometer system, receiving the excited light or the reflected light, resolving the wavebands of the received light into spectra and analyzing the spectra to obtain the spectrum data thereof; and a transmitter or a storage device, sending out or storing the spectrum data.

To enable the objectives, technical contents, characteristics, and accomplishments of the present invention to be more easily understood, the embodiments of the present invention are to be described below in detail in cooperation with the attached drawings.

### BRIEF DESCRIPTION OF THE DRAWINGS

FIG. 1 is a sectional view schematically showing the capsule of a conventional in vivo video camera system.

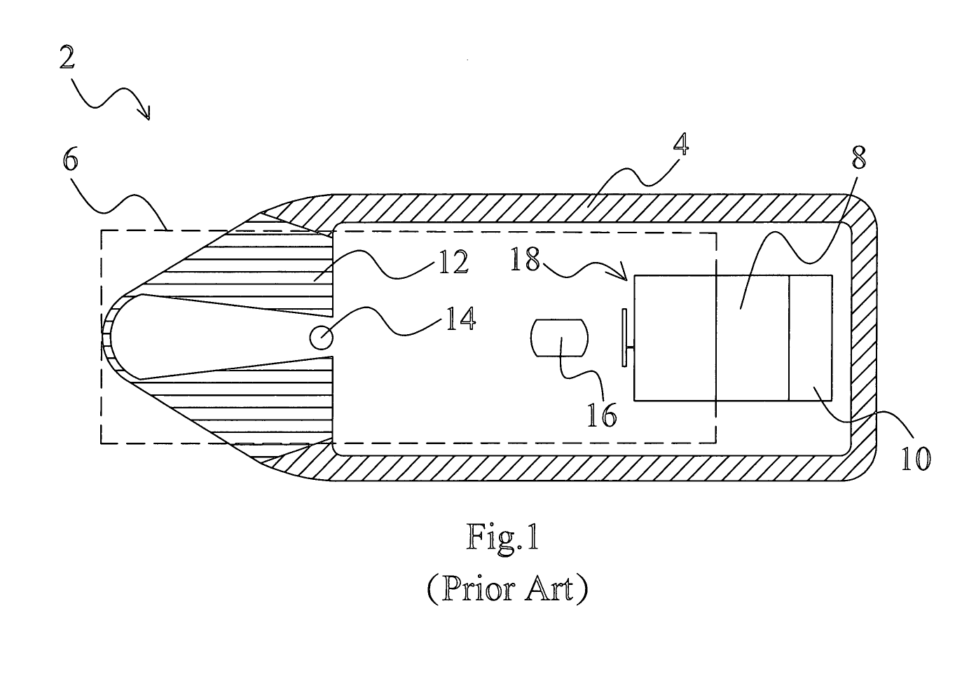

FIG. 2 is a diagram schematically showing the architecture of the system according to one embodiment of the present invention.

FIG. 3 is a diagram schematically showing the architecture of the system according to one embodiment of the present invention.

FIG. 4 is a sectional view schematically showing the placed-in type capsular spectrometric endoscope according to one embodiment of the present invention.

FIG. 5 is a sectional view schematically showing the placed-in type capsular spectrometric endoscope according to on embodiment of the present invention.

FIG. 6 is a sectional view schematically showing the placed-in type capsular spectrometric endoscope according to one embodiment of the present invention.

FIG. 7 is a sectional view schematically showing the placed-in type capsular spectrometric endoscope according to one embodiment of the present invention.

### DETAILED DESCRIPTION OF THE INVENTION

The present invention is an in vivo inspection/analysis system, wherein a light source is used to illuminate a inspected tissue in vivo, such as the tissue of the digestive system; the test tissue will generate a spectrum, and the spectrum of the tissue will be used to implement an in vivo inspection and the analysis thereof.

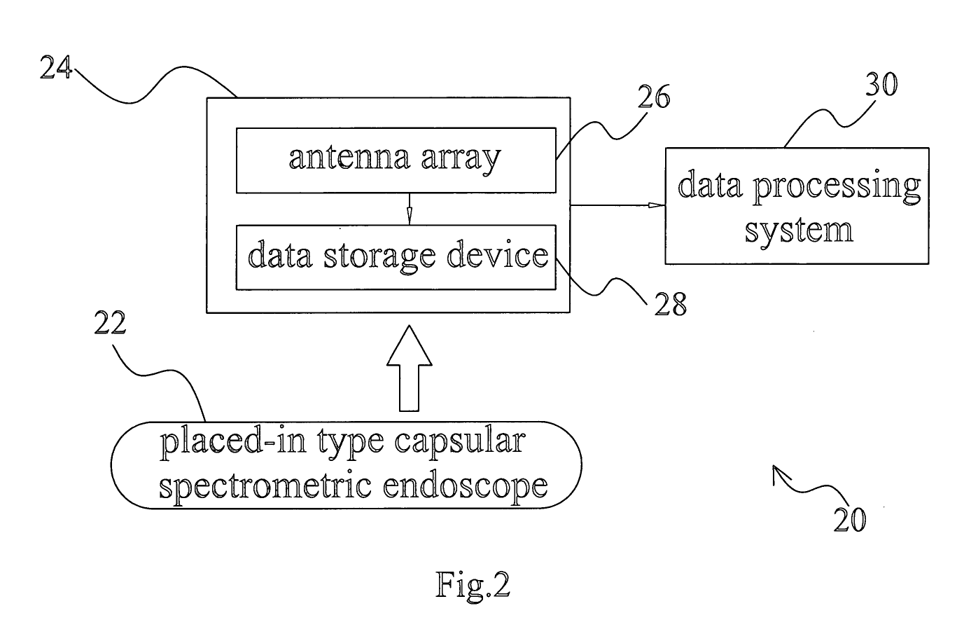

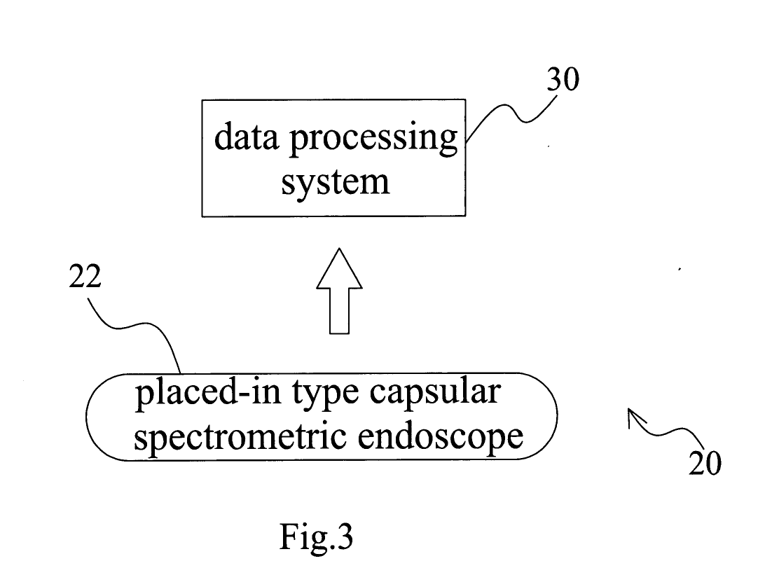

Refer to FIG. 2 and FIG. 3 diagrams schematically showing the architectures of the systems according to two embodiments of the present invention. As shown in the diagrams, the in vivo spectrometric inspection system 20 of the present invention comprises a placed-in type capsular spectrometric endoscope 22; the spectrum data obtained by the placed-in type capsular spectrometric endoscope 22 may be sent out to the exterior of the testee body for data analysis via the following two methods. In the first method, as shown in FIG. 2, the spectrum data sent out by the placed-in type capsular spectrometric endoscope 22 is received by a receiving system 24 installed externally, and the receiving system 24 comprises: an antenna array 26, consisting of multiple antennas, and used to receive the spectrum data; and a data storage device 28, coupled to the antenna array 26, and storing the spectrum data received by the antenna array 26. Besides, a data processing system 30 is coupled to the receiving system 24 and used to process and analyze the spectrum data received by the antenna array 26, wherein the data processing system 30 can process the spectrum data directly coming from the antenna array 26 and the spectrum data coming from the data storage device 28. In the second method, as shown in FIG. 3, the spectrum data is stored in the placed-in type capsular spectrometric endoscope 22; after the placed-in type capsular spectrometric endoscope 22 is excreted from the testee body, the spectrum data is read and transmitted to the data processing system 30 for the succeeding analysis.

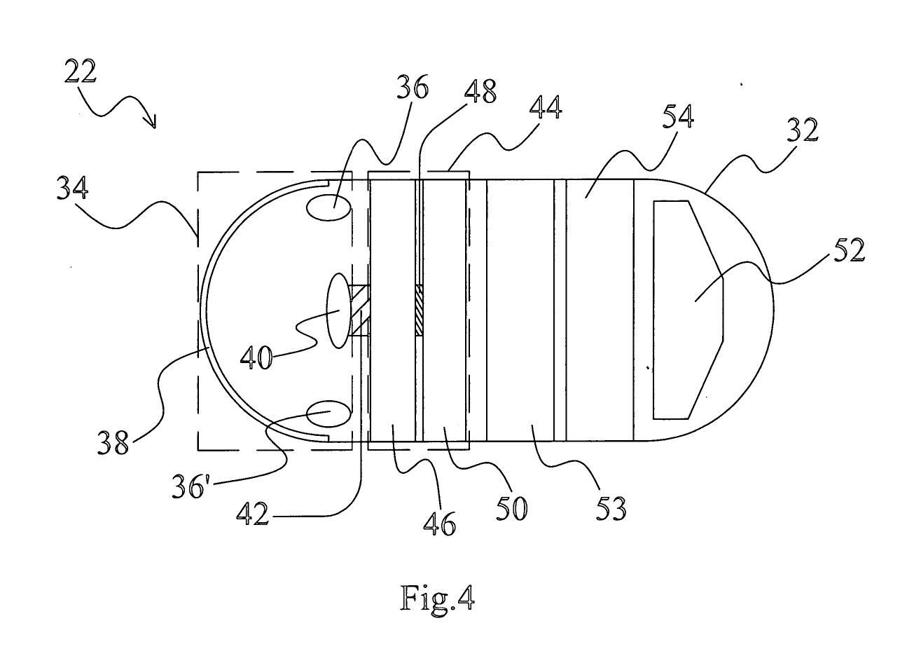

Refer to FIG. 4 a sectional view schematically showing the placed-in type capsular spectrometric endoscope according to one embodiment of the present invention. As shown in the diagram, the placed-in type capsular spectrometric endoscope 22 comprises a swallowable capsule 32, and an optical system 34 is installed inside the swallowable capsule 32 and has two light-source generating devices 36 and 36′. The light sources created by the light-source generating devices 36 and 36′ pass through a transparent optical protective cover 38 and illuminate an in vivo tissue. The light source hits the tissue to excite a light, or the light source is partially absorbed and partially reflected by the tissue; a light-receiving device 40 receives the excited light or the reflected light; the received light is transmitted to a spectrometer system 44 via a light-transmission device 42, such as a lens or a set of optical fibers. In the spectrometer system 44, a beam-splitting device 46 sequentially resolves the wavebands of the received light. The resolved wavebands are transmitted to at least one spectrum detection/analysis device 50 for spectral analysis via a transmission device 48. The spectrum data obtained by the spectrometer system 44 is sent out to the exterior of the testee body via a data-transmission device 52, and received by an external receiving system. Otherwise, the spectrum data may also be stored in a storage device 53 of the placed-in type capsular spectrometric endoscope 22; in this case, the spectrum data will be carried out when the placed-in type capsular spectrometric endoscope 22 is excreted. Besides, the placed-in type capsular spectrometric endoscope 22 also has a battery device 54 for power supply.

The light-receiving device 40 may be a lens, a set of lenses, or another optical element. The light-source-generating device 36 and 36′ may be light-emitting diodes, laser diodes, incandescent lamps or other light-emitting devices. The light source generated by the light-source generating device 36 and 36′ is a wide-spectrum light source, including: ultraviolet light, visible light, and infrared light, which may be synthesized with multiple light sources of specified wavebands, one or multiple swept-band light sources, or one or multiple light sources respectively having a single wide-spectrum waveband. The light-source generating device 36 and 36′ may utilize filters, the gradient change of a film coating, the change of incident angles, optical gratings, photon crystals, or a Fabry-Perot method to generate light sources. The beam-splitting device 46 of the spectrometer system 44 may utilize filters, the gradient change of a film coating, the change of incident angles, micro optical gratings, photon crystals, or Fabry-Perot elements to disperse the wavebands of the excited light or the reflected light. The spectrum detection/analysis device 50 may be a CMOS (Complementary Metal Oxide Semiconductor) sensor, a CCD (Charge Coupled Device) sensor, or another optoelectronic sensor. The transmission device 48 may be a set of optical fibers or another optical device.

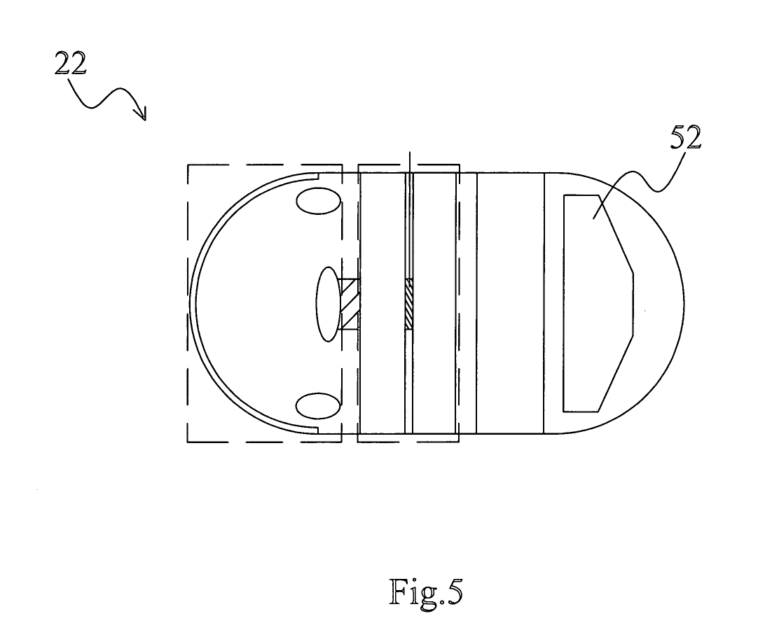

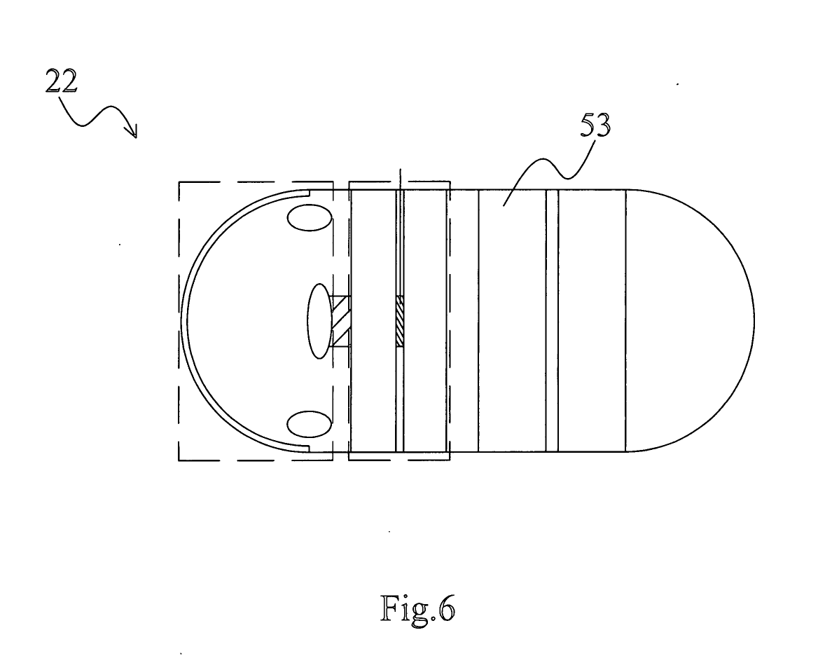

The phenomenon that a nidus responds differently to light sources of different wavelengths, such as the autofluorescence of a tissue illuminated by ultraviolet light, can be used by the in vivo spectrometric inspection system 20 of the present invention to inspect the digestive system of a living body. In this case, after the placed-in type capsular spectrometric endoscope 22 has been swallowed by the testee and entered into the digestive system, the light-source generating device 36 and 36′ of the optical system 34 generates a wide-spectrum light source to illuminate the tissue of the digestive system; the light source hitting the tissue may excite a light, or the light source may be partially absorbed and partially reflected by the tissue. The light excited on the tissue or the light reflected by the tissue will pass through the optical protective cover 38 and be received by the light-receiving device 40 and then be transmitted to the spectrometer system 44 via the light-transmission device 42. The light transmitted to the spectrometer system 44 is a full-waveband light source, and the beam-splitting device 46 of the spectrometer system 44 will resolve the full-waveband light source into spectra of different wavebands, such as wavebands λ₁, λ₂ . . . , λₘ. The spectra of different wavebands are partitioned and sent into the spectrum detection/analysis device 50 for spectral analysis in order to obtain a spectrum data. The data-transmission device 52 continuously or discontinuously sends out the spectrum data acquired by the spectrometer system 44 to the exterior of the testee body. In the exterior of the testee body, the antenna array 26 of the receiving system 24 receives the spectrum data continuously or discontinuously sent out by the data-transmission device 52. Otherwise, the spectrum data may also be stored in a storage device 53 of the placed-in type capsular spectrometric endoscope 22, and the spectrum data will be carried out when the placed-in type capsular spectrometric endoscope 22 is excreted. After the spectrum data has been obtained in the exterior of the testee body via the abovementioned methods, the spectrum data will be analyzed by the data processing system 30 to obtain the responses of the inspected tissue to different wavebands at a certain time. The data of an identical waveband at different time are assembled together, and then, a continuous full-spectrum data can be obtained via integrating the data of all the concerned wavebands for the full test duration. It is not necessary for the present invention that the placed-in type capsular spectrometric endoscope 22 simultaneously possesses both the data-transmission device 52 and the storage device 53. Refer to FIG. 5 and FIG. 6 diagrams schematically showing the placed-in type capsular spectrometric endoscope according to two embodiments of the present invention. In the present invention, the placed-in type capsular spectrometric endoscope 22 may also possess only one of the data-transmission device 52 and the storage device 53.

When the placed-in type capsular spectrometric endoscope 22 adopts a wide-spectrum light source, an outgrowth tissue may be illuminated with a short-wavelength light thereof to obtain the spectrum of the autofluorescence emitted by the outgrowth tissue. The autofluorescence spectrum is analyzed to obtain the continuous variation of the concerned wavebands so that the tissue of an early-stage cancer can be distinguished. In comparison with the white-light image obtained by a white-light inspection, the present invention does not have to obtain the image of an in vivo tissue but utilizes the spectrum data of the in vivo tissue to identify various abnormal tissues. Therefore, the present invention can promote the discrimination rate of cytopathic effect or early-stage cancer tissues, which are hard to find with naked eyes.

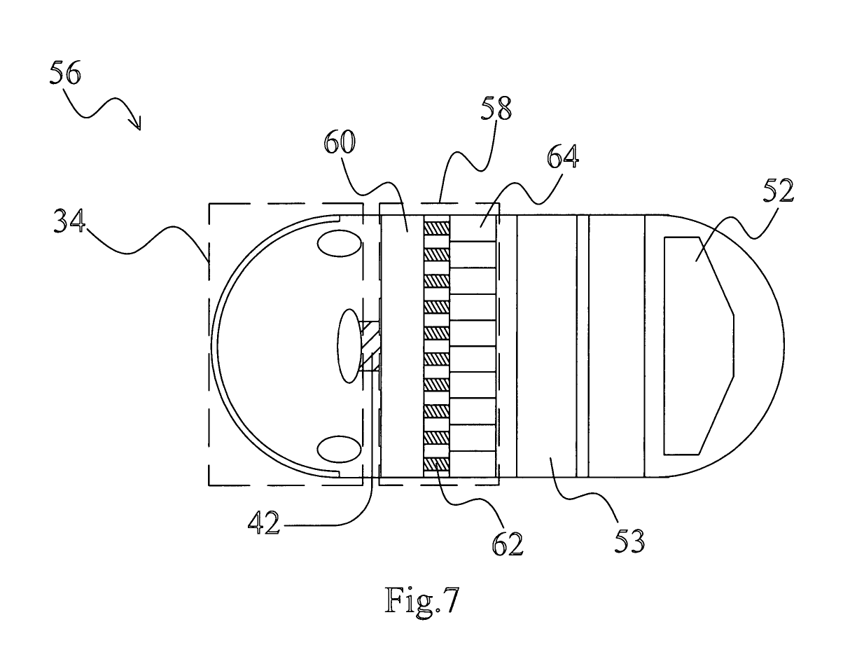

Refer to FIG. 7 a diagram schematically showing a placed-in type capsular spectrometric endoscope according to one embodiment of present invention, wherein the spectrometer system shown in FIG. 4 is modified. In this embodiment, the placed-in type capsular spectrometric endoscope 56 is swallowed, and an optical system 34 generates light sources to illuminate an in vivo tissue. The light source hits the tissue to excite a light, or the light source is partially absorbed and partially reflected by the tissue. The optical system 34 receives the excited light or the reflected light, and a light-transmission device 42 transmits the received light to a spectrometer system 58. In the spectrometer system 58, a beam-splitting device 60 resolves the wavelength components of the received light into spectra of different wavebands, such as wavebands λ₁₁, λ₂₁ . . . , λₘₙ. The resolved wavebands are transmitted to multiple spectrum detection/analysis devices 64 for spectral analysis via multiple transmission devices 62, such as a set of optical fibers. The spectrum data obtained by each spectrum detection/analysis device 64 is sent out to the exterior of the testee body via a data-transmission device 52. Otherwise, the spectrum data may also be stored in a storage device 53. The spectrum data will be further analyzed by a data processing system to obtain different-waveband data of a same area. The rest of this embodiment is similar to that mentioned in the descriptions of from FIG. 2 to FIG. 4 and will not be repeated here.

As nidus respond differently to light sources of different wavelengths, the present invention utilizes a wide-spectrum light source to illuminate in vivo tissues, and the in vivo tissues will generate spectra, and the spectrum data thereof will be analyzed to obtain the spectral responses of various abnormal cells. The present invention not only can solve the problems that naked eyes are hard to identify a cell abnormality, and that the resolving power of an early-stage cancer tissue is inferior in conventional inspection technologies, but also can promote the accuracy of medical inspection via providing an in vivo spectrometric inspection system to obtain a series of in vivo spectrum data and the spectral responses of various abnormal cells.

Those embodiments described above are to clarify the characteristics of the present invention in order to enable the persons skilled in the art to understand, make, and use the present invention; however, it is not intended to limit the scope of the present invention, and any equivalent modification and variation according to the spirit of the present invention is to also included within the scope of the claims stated below.

What is claimed is:

1. An in vivo spectrometric inspection system, comprising:
a swallowable capsule, further comprising:
an optical system, generating a light source to illuminate a test object, and receiving the light excited by said light source or the light reflected from said test object;
a spectrometer system, receiving the light excited by said light source or the light reflected by said test object, and performing a beam-splitting process and an analysis to obtain a spectrum data; and
a data-transmission device, transmitting said spectrum data output by said spectrometer system; and
a receiving system, receiving said spectrum data.

2. The in vivo spectrometric inspection system according to claim 1, wherein said spectrometer system further comprises:
a beam-splitting device, receiving said excited light or said reflected light, and resolving the wavebands of said excited light or said reflected light into spectra of different wavebands, wherein said beam-splitting device utilizes filters, the gradient change of a film coating, the change of incident angles, micro optical gratings, photon crystals, or Fabry-Perot elements to disperse the wavebands of said excited light or said reflected light; and
at least one spectrum detection/analysis device, coupled to said beam-splitting device, and analyzing said spectra of different wavebands to obtain said spectrum data, wherein said spectrum detection/analysis device may be a CMOS (Complementary Metal Oxide Semiconductor) sensor, a CCD (Charge Coupled Device) sensor, or another optoelectronic sensor.

3. The in vivo spectrometric inspection system according to claim 2, further comprising a transmission device, which transmits said spectra of different wavebands dispersed by said beam-splitting device to said spectrum detection/analysis device, wherein said transmission device is an optical element and said optical element is a set of optical fibers.

4. The in vivo spectrometric inspection system according to claim 1, wherein said optical system further comprising:
an optical protective cover;
at least one light-source generating device, generating said light source, wherein said light-source generating device may be light-emitting diodes, laser diodes, incandescent lamps or other light-emitting devices or may utilize filters, the gradient change of a film coating, the change of incident angles, optical gratings, photon crystals, or a Fabry-Perot method to generate said light source; and
a light-receiving device, receiving said excited light or said reflected light, wherein said light-receiving device is an optical element and said optical element may be a lens or a set of lenses.

5. The in vivo spectrometric inspection system according to claim 1, wherein said light source is a wide-spectrum light source, and said wide-spectrum light source further comprises: ultraviolet light, visible light and infrared light.

6. The in vivo spectrometric inspection system according to claim 1, wherein said light source may be synthesized with multiple light sources of specified wavebands, one or multiple swept-band light sources, or one or multiple light sources respectively having a single wide-spectrum waveband.

7. The in vivo spectrometric inspection system according to claim 1, further comprising a light-transmission device, which transmits said excited light or said reflected light received by said optical system to said spectrometer system, wherein said light-transmission device is a set of optical fibers.

8. The in vivo spectrometric inspection system according to claim 1, further comprising a data-processing system, which performs a data analysis on said spectrum data received by said receiving system, and said receiving system further comprises:
an antenna array, further comprising multiple antennas, and used to receive said spectrum data; and
a data storage device, coupled to said antenna array, and storing said spectrum data received by said antenna array.

9. An in vivo spectrometric inspection system, comprising:
a swallowable capsule, further comprising:
an optical system, generating a light source to illuminate a test object, and receiving the light excited by said light source or the light reflected from said test object;
a spectrometer system, receiving the light excited by said light source or the light reflected by said test object, and performing a beam-splitting process and an analysis to obtain a spectrum data; and
a storage device, storing said spectrum data; and
a data-processing system, receiving said spectrum data stored in said storage device, and performing a data analysis on said spectrum data.

10. The in vivo spectrometric inspection system according to claim 9, wherein said spectrometer system further comprises:
a beam-splitting device, receiving said excited light or said reflected light, and resolving the wavebands of said excited light or said reflected light into spectra of different wavebands, wherein said beam-splitting device utilizes filters, the gradient change of a film coating, the change of incident angles, micro optical gratings, photon crystals, or Fabry-Perot elements to disperse the wavebands of said excited light or said reflected light; and
at least one spectrum detection/analysis device, coupled to said beam-splitting device, and analyzing said spectra of different wavebands to obtain said spectrum data, wherein said spectrum detection/analysis device may be a CMOS (Complementary Metal Oxide Semiconductor) sensor, a CCD (Charge Coupled Device) sensor, or another optoelectronic sensor.

11. The in vivo spectrometric inspection system according to claim 10, further comprising a transmission device, which transmits said spectra of different wavebands dispersed by said beam-splitting device to said spectrum detection/analysis device, wherein said transmission device is an optical element and said optical element is a set of optical fibers.

12. The in vivo spectrometric inspection system according to claim 9, wherein said optical system further comprising:
an optical protective cover;
at least one light-source generating device, generating said light source, wherein said light-source generating device may be light-emitting diodes, laser diodes, incandescent lamps or other light-emitting devices or may utilize filters, the gradient change of a film coating, the change of incident angles, optical gratings, photon crystals, or a Fabry-Perot method to generate said light source; and
a light-receiving device, receiving said excited light or said reflected light, wherein said light-receiving device is an optical element and said optical element may be a lens or a set of lenses.

13. The in vivo spectrometric inspection system according to claim 9, wherein said light source is a wide-spectrum light source and said wide-spectrum light source further comprises: ultraviolet light, visible light and infrared light.

14. The in vivo spectrometric inspection system according to claim 9, wherein said light source may be synthesized with multiple light sources of specified wavebands, one or multiple swept-band light sources, or one or multiple light sources respectively having a single wide-spectrum waveband.

15. The in vivo spectrometric inspection system according to claim 9, further comprising a light-transmission device, which transmits said excited light or said reflected light received by said optical system to said spectrometer system, wherein said light-transmission device is a set of optical fibers.

16. A placed-in type capsular spectrometric endoscope, comprising:
a swallowable capsule, further comprising:
an optical system, generating a light source to illuminate a test object, and receiving the light excited by said light source or the light reflected from said test object;
a spectrometer system, receiving the light excited by said light source or the light reflected by said test object, and performing a beam-splitting process and an analysis to obtain a spectrum data;
a storage device, storing said spectrum data; and
a data-transmission device, transmitting said spectrum data output by said spectrometer system to a receiving system.

17. The placed-in type capsular spectrometric endoscope according to claim 16, wherein said spectrometer system further comprises:
a beam-splitting device, receiving said excited light or said reflected light, and resolving the wavebands of said excited light or said reflected light into spectra of different wavebands, wherein said beam-splitting device utilizes filters, the gradient change of a film coating, the change of incident angles, micro optical gratings, photon crystals, or Fabry-Perot elements to disperse the wavebands of said excited light or said reflected light;
at least one spectrum detection/analysis device, coupled to said beam-splitting device, and analyzing said spectra of different wavebands to obtain said spectrum data, wherein said spectrum detection/analysis device may be a CMOS (Complementary Metal Oxide Semiconductor) sensor, a CCD (Charge Coupled Device) sensor, or another optoelectronic sensor; and
a transmission device, which transmits said spectra of different wavebands dispersed by said beam-splitting device to said spectrum detection/analysis device, wherein said transmission device is an optical element and said optical element is a set of optical fibers.

18. The placed-in type capsular spectrometric endoscope according to claim 16, wherein said optical system further comprising:
an optical protective cover;
at least one light-source generating device, generating said light source, wherein said light-source generating device may be light-emitting diodes, laser diodes, incandescent lamps or other light-emitting devices or may utilize filters, the gradient change of a film coating, the change of incident angles, optical gratings, photon crystals, or a Fabry-Perot method to generate said light source; and
a light-receiving device, receiving said excited light or said reflected light, wherein said light-receiving device is an optical element and said optical element may be a lens or a set of lenses.

19. The placed-in type capsular spectrometric endoscope according to claim 16, wherein said light source is a wide-spectrum light source, wherein said wide-spectrum light source further comprises: ultraviolet light, visible light and infrared light or may be synthesized with multiple light sources of specified wavebands, one or multiple swept-band light sources, or one or multiple light sources respectively having a single wide-spectrum waveband.

20. The placed-in type capsular spectrometric endoscope according to claim 16, further comprising a light-transmission device, which transmits said excited light or said reflected light received by said optical system to said spectrometer system, wherein said light-transmission device is a set of optical fibers.
